(19) United States
(12) Patent Application Publication
(10) Pub. No.: US 2026/0085993 A1
(43) Pub. Date: Mar. 26, 2026

(54) LEAK DETECTION USING REFLECTION-BASED IMAGING

(71) Applicant: GM GLOBAL TECHNOLOGY OPERATIONS LLC, Detroit, MI (US)

(72) Inventors: Hassan Ghassemi-Armaki, Northville, MI (US); Blair E. Carlson, Ann Arbor, MI (US); Randolph Zeitvogel, Beverly Hills, MI (US); Ranajit Ghosh, Vernon Hills, IL (US); Andrew C. Bobel, Troy, MI (US); Jeffrey A. Abell, Rochester Hills, MI (US)

(73) Assignee: GM GLOBAL TECHNOLOGY OPERATIONS LLC, Detroit, MI (US)

(21) Appl. No.: 18/897,098

(22) Filed: Sep. 26, 2024

(51) Int. Cl. G01M 3/38; G01N 33/00

(52) U.S. Cl. CPC G01M 3/38; G01N 33/0031

(57) ABSTRACT

A leak detection system includes an emitter, detector, and electronic control unit (ECU). The emitter directs electromagnetic energy having a predetermined wavelength range, e.g., infrared energy, toward a surface of a product. The product defines an enclosure chamber containing a trace gas. The detector is positioned between the emitter and the product at an offset distance from the surface. The detector detects reflected energy. The ECU receives a signal from the detector that is indicative of a spectrum of the reflected energy and identifies a detected leak in the product. This includes comparing the spectrum of the reflected energy to a predetermined spectrum of the trace gas. The trace gas has a wavelength within the predetermined wavelength range of the emitter. The ECU generates an output signal in response to the leak, with the output signal identifying a presence and location of the leak.

### INTRODUCTION

Leak testing of a manufactured product may involve the introduction of an inert gas into a void volume of the product, e.g., an internal cavity, chamber, fluid channel, or tube. The gas is carefully sealed within the chamber. Leaks are then detected using a number of different approaches. For example, decay of pressure within the void volume may be monitored over time, with the detected pressure decay possibly indicating the presence of a leak. Another technique referred to as “helium sniffing” involves filling the void volume with pressurized helium gas. A mass spectrometer is then used to detect the presence of helium in the surrounding air. While effective in some applications, these and other leak detection techniques remain suboptimal when used to perform leak testing with high repeatability and accurate resolution of the leak's location.

### SUMMARY

A reflection-based system and method are described below for accurately detecting and locating a leak point in a product defining a void internal volume. During leak testing, this volume or enclosure chamber may be filled with an application-suitable trace gas such as carbon dioxide or helium. While myriad consumer, transportation, and industrial products exist that have such an enclosure chamber, products used in a non-limiting battery application include a battery tray, a welded cold plate, a battery cover, battery pack, battery cell, and various other objects of interest. Other non-battery vehicular products that would benefit from the present teachings leak testing and leak location include internal combustion engines, heat exchangers, etc., with a host of non-vehicular products likewise benefiting. In the event the enclosure chamber experiences a leak, some amount of the aforementioned trace gas will escape to the surrounding atmosphere. The reflection-based leak testing solutions presented herein are therefore directed to detecting the presence of such a leak while accurately locating underlying leak points. Subsequent corrective actions may be taken as needed in response to detecting and locating the leak.

In particular, a leak detection system in accordance with a representative embodiment includes an emitter, a detector, and an electronic control unit (ECU), with “a” and “an” meaning “at least one” or “one or more” unless otherwise specified. During a leak test, the emitter, for instance an infrared (IR) emitter or array thereof, directs electromagnetic energy toward a surface of a product, with the energy having a predetermined wavelength range. The product as contemplated herein defines the above-noted enclosure chamber, which in turn is filled with a desired trace gas when conducting the leak test. The detector, which is configured to detect reflected energy from the product/surface thereof, is positioned between the emitter and the product at an application-specific offset distance from the product's surface.

The ECU in this exemplary configuration receives an electronic input signal from the detector. The electronic input signal is indicative/descriptive of a spectrum of the reflected energy, and in particular the detected wavelength/wavelengths thereof. The ECU also identifies a detected leak in the product by comparing the spectrum of the reflected energy to a predetermined spectrum of the trace gas. The trace gas has a wavelength that falls within the predetermined wavelength range of energy from the emitter. In one or more embodiments, the predetermined wavelength range is about 2 microns (μ) to about 10μ, with other possible wavelength ranges being usable in other applications of the present teachings.

The ECU also generates an electronic output signal in response to the detected leak. The electronic output signal identifies both a presence and a location of the leak. Such information is usable by the ECU and/or production operators/maintenance personnel, for instance to correct the leak or perform a root cause analysis. The ECU in one or more implementations may detect the presence and location of the leak by analyzing a difference in contrast between the spectrum of the reflected energy and the predetermined spectrum of the trace gas.

For some constructions of the product, including exemplary battery components for use in a battery electric vehicle (BEV), a hybrid electric vehicle (HEV), a plug-in HEV (PHEV), or another vehicle or mobile system, the predetermined offset distance from the surface of the product may be about 0.25 meters (m) to about 5 m, i.e., about 10-200 inches.

The detector may include an image filter having a bandwidth encompassing the wavelength of the trace gas and energy from the emitter. The emitter, which may be stationary or moveable with respect to the product in different implementations, may include an array of emitters positioned or arranged in proximity to the product. In such an embodiment, each respective emitter of the array of emitters is configured to illuminate the product from a different angle.

The leak detection system in one or more embodiments may include a robot and/or an overhead gantry system (“gantry”). The detector may be connected to the robot, with the robot being configured to move the detector with respect to the product. The gantry may be used to position the emitter relative to the product in this embodiment or other embodiments. A three-dimensional (3D) laser scanner, optionally connectable to the gantry, may also be used as part of the leak detection system to scan the surface of the product and output a 3D scan file indicative of a contour of the surface. The ECU in such an embodiment may compare the contour of the surface to a calibrated baseline contour to ascertain a surface distortion level of the product. The ECU may thereafter generate the electronic output signal in part by using the surface distortion level.

A leak detection method is also described herein. An embodiment of such a method includes using an emitter to direct electromagnetic energy of a predetermined wavelength range toward a surface of the product. The method may include detecting reflected energy via the detector, which as noted above is positioned between the emitter and the product at an offset distance from the product's surface. As part of the method, the ECU receives the electronic input signal from the detector, with the signal being indicative of a spectrum of the reflected energy. The method additionally includes identifying a detected leak in the product via the ECU. This action may include comparing the spectrum of the reflected energy to a predetermined spectrum of the trace gas. The trace gas for its part has a wavelength that falls within the predetermined wavelength range of the emitter. The method further includes generating the electronic output signal in response to the detected leak, with the output signal identifying a presence and location of the leak.

The leak detection system in accordance with another disclosed embodiment includes an IR emitter array configured to direct beams of IR energy toward a surface of a product in a wavelength range of about 2μ to about 10μ, with the product defining the above-mentioned enclosure chamber. In this particular embodiment, the chamber contains carbon dioxide as the trace gas. An IR detector array is positioned between the IR emitter array and the product at an offset distance of less than about 5 m from the surface of the product. The IR detector array is configured to detect reflected IR energy during leak testing of the product. The 3D laser scanner and the ECU are also used as part of this non-limiting embodiment.

The ECU is configured to receive an electronic input signal from the IR detector array, with the input signal being indicative of a spectrum of the reflected IR energy. The ECU also commands the 3D scanner to generate a 3D scan file indicative of a contour of the surface and compares the contour of the surface to a calibrated baseline contour to ascertain a surface distortion level of the product. Additionally, the ECU identifies a detected leak in the product using the surface distortion and by comparing the spectrum of the reflected energy to a predetermined spectrum of the trace gas. The ECU ultimately generates an electronic output signal in response to the detected leak, the output signal identifying a presence and location of the leak.

The above features and advantages, and other features and attendant advantages of this disclosure, will be readily apparent from the following detailed description of illustrative examples and modes for carrying out the present disclosure when taken in connection with the accompanying drawings and the appended claims. Moreover, this disclosure expressly includes combinations and sub-combinations of the elements and features presented above and below.

### BRIEF DESCRIPTION OF THE DRAWINGS

FIG. 1 is an illustration of a reflection-based leak detection system constructed as set forth herein.

FIG. 1A illustrates a portion of the leak detection system in accordance with a possible implementation.

FIGS. 2 and 3 illustrate alternative constructions of the leak detection system of FIGS. 1 and 1A.

FIG. 4 is a flow chart illustrating a robot-assisted leak detection method in accordance with an aspect of the present disclosure.

The present disclosure may be modified or embodied in alternative forms, with representative embodiments shown in the drawings and described in detail below. Inventive aspects of the present disclosure are not limited to the disclosed embodiments. Rather, the present disclosure is intended to cover alternatives falling within the scope of the disclosure as defined by the appended claims.

### DETAILED DESCRIPTION

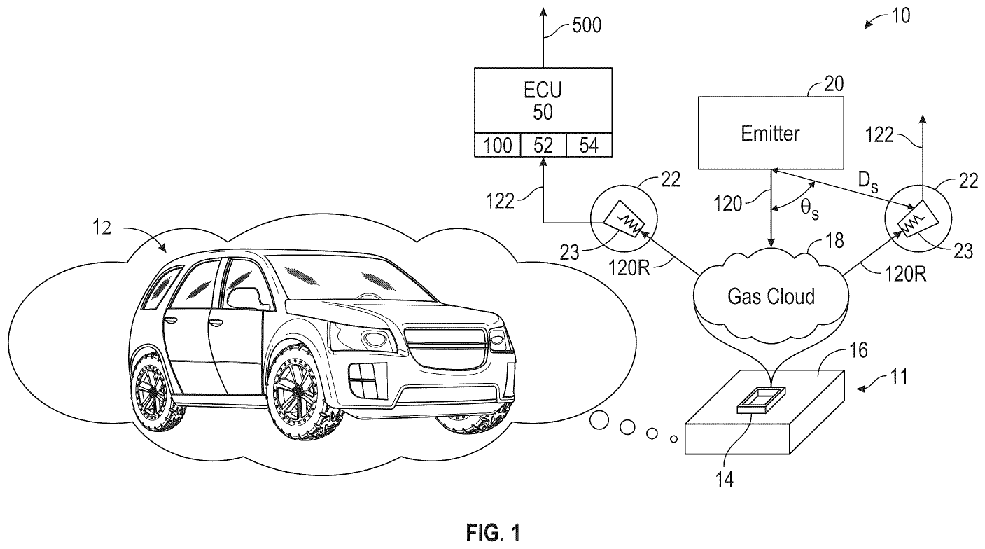

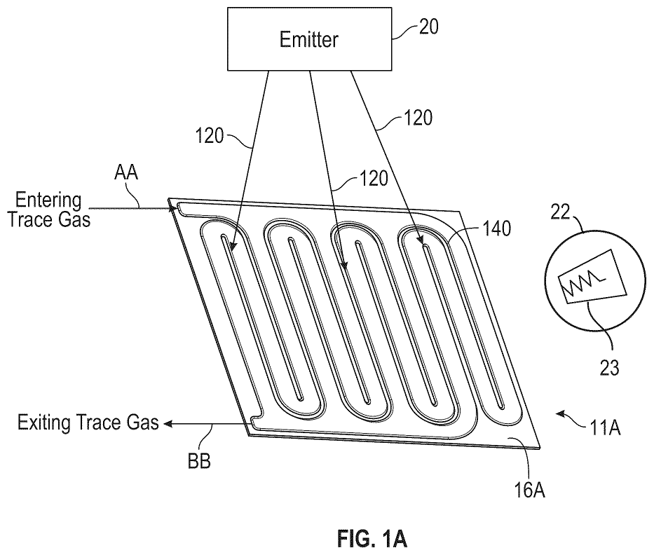

Referring to the drawings, wherein like reference numbers refer to like features throughout the several views, FIG. 1 illustrates a leak detection system 10 operable for accurately detecting and locating a point of leakage in a product 11. In a non-limiting use scenario, the product 11 may be a component part of a vehicle 12. For instance, the vehicle 12 may be embodied as a battery electric vehicle (BEV), a plug-in hybrid electric vehicle (PHEV), a hybrid electric vehicle (HEV), an extended range electric vehicle (EREV), or another vehicle or other mobile system. In such an embodiment, the product 11 may include a vehicle component such as a battery tray, a welded cold plate, a battery cover, or another component of a vehicle battery. However, the product 11 may include vehicular or non-vehicular components that require leak testing and leak location as set forth herein, for instance internal combustion engines, heat exchangers, etc. Other embodiments of the product 11 therefore may be contemplated within the scope of the disclosure, and therefore vehicular and mobile embodiments as described herein are merely representative of the present teachings and non-limiting thereof.

The product 11 of FIG. 1 has a surface 16 and defines an enclosure chamber 14. The enclosure chamber 14 may be variously embodied as a void volume in the form of, e.g., a cavity, tube, reservoir, etc. During leak testing of the product 11, the enclosure chamber 14 is filled with an application suitable trace gas 18 such that the enclosure chamber 14 contains the trace gas 18 therein during leak testing. The composition of the trace gas 18 may vary in accordance with the intended application. Example inert gas compositions include carbon dioxide (CO₂) and helium, without limitation.

The leak detection system 10 of FIG. 1 is based on the principle of energy reflection and imaging of reflected energy spectra from the product 11 and/or escaping gas clouds of the trace gas 18. To that end, the leak detection system 10 includes an emitter 20 configured to direct light or other electromagnetic radiation, hereinafter emitted energy 120, of a predetermined wavelength range toward the surface 16 of the product 11, variously as a single beam, multiple beams, or a scanning beam. In one or more representative embodiments, the emitted energy 120 may be infrared (IR) energy, and the emitter 20 may include an IR emitter. As used herein, the IR spectrum may encompass energy having a wavelength of about 750 nanometers (nm) to about 1.4 microns (μ), i.e., near IR, energy having a wavelength of about 1.4μ to about 3μ (mid-IR), and/or energy having a wavelength of about 3μ to about 1 millimeter (mm), i.e., far IR. Non-IR wavelengths of the emitted energy 120 may be used in other implementations, for example visible light, ultraviolet energy, etc., and therefore infrared implementations are illustrative of aspects of the present teachings and are non-limiting thereof. When the emitted energy 120 includes IR energy, the predetermined wavelength range of the emitted energy 120 from the emitter 20 may be about 2μ to about 10μ, once again without limiting the present teachings to such a range.

Additionally, the leak detection system 10 includes at least one detector 22. Each detector 22 is positioned between the emitter 20 and the product 11 at an offset distance (D_{S}) from the surface 16. The offset distance may vary within the scope of the disclosure depending on the construction of the product 11, with an offset distance of about 0.25 meters (m) to about 5 m (about 10 inches to about 200 inches) being possible in accordance with an embodiment. When the product 11 is constructed as the battery tray as noted above, an optimal standoff distance may be about 1.1 m to about 1.65 m (about 45 inches to about 65 inches). The emitter 20 is also arranged at an offset angle (θ_{S}) relative to the detector 22, with the particular offset angle varying with the intended application and number of detectors 22 used in the construction of the leak detection system 10.

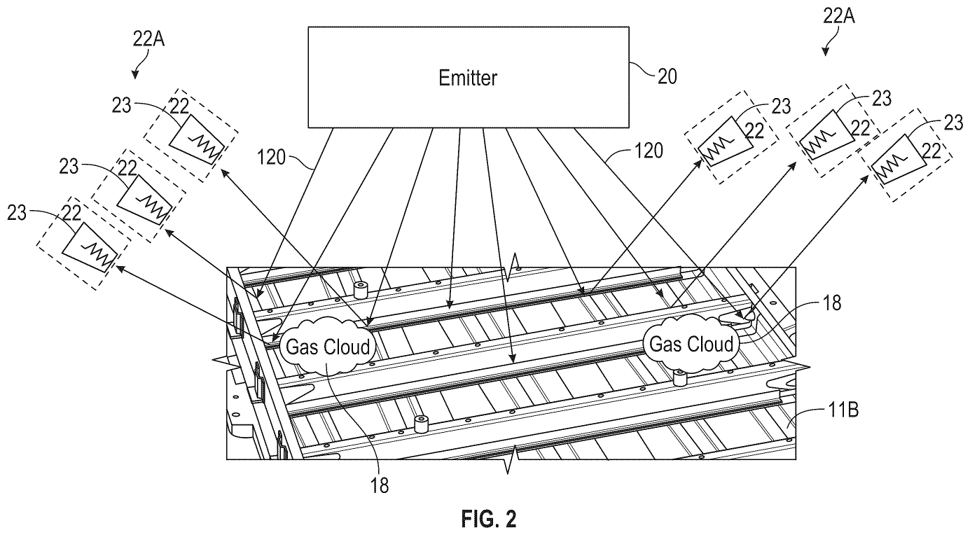

The detector 22 shown in FIG. 2 is configured to detect reflected energy 120R during leak testing of the product 11, with the reflected energy 120R returning to the detector 22 when reflected off of the product 11 and/or a gas cloud composed of the trace gas 18. Although the detector 22 is illustrated as a pair of detectors 22 in FIG. 1 for illustrative simplicity, more or fewer detectors 22 may be used in other implementations as described below. Each detector 22 may include a filter 23 having a bandwidth encompassing the wavelength of the trace gas 18 and the reflected energy 120R, thus admitting such wavelengths and blocking others.

Additionally, the respective detectors 22 as used herein are configured to transmit an electronic input signal 122 to an electronic control unit (ECU) 50 in accordance with the disclosure. The ECU 50 is in communication with the detector 22, wirelessly and/or via physical transfer conductions, and thus is configured to receive the electronic input signal 122 from the detector 22. The electronic input signal 122 for its part is an electrical signal indicative or descriptive of a spectrum of the reflected energy 120R.

The ECU 50 in the various embodiments described below is equipped in hardware and programmed in software, i.e., configured, to identify a detected leak in the product 11. The ECU 50 may do so by comparing the spectrum of the reflected energy 120R to a predetermined spectrum of the trace gas 18, for instance one that has been previously stored in memory 54 of the ECU 50. The ECU 50 may also be configured to detect the presence and the location of the leak by analyzing a difference in contrast between the spectrum of the reflected energy 120R and the predetermined spectrum of the trace gas 18. The trace gas 18 in turn has a wavelength that falls within the predetermined wavelength range of the detector 22. The ECU 50 is also configured to generate an output signal 500 in response to a detected leak, with the output signal 500 identifying a presence and location of the leak.

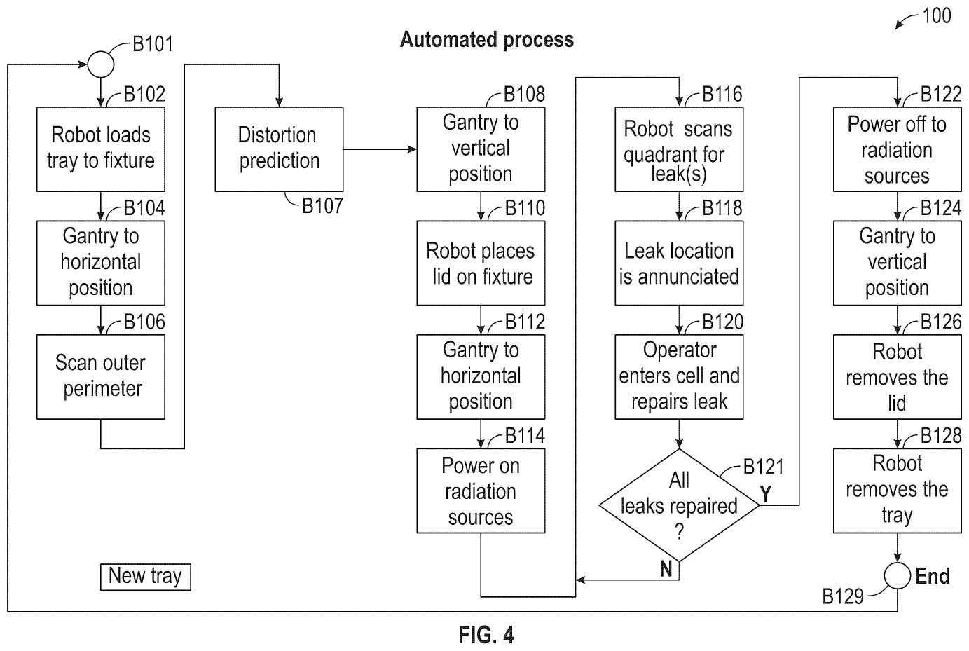

The ECU 50 may be implemented as one or more computer devices, and thus includes hardware in the form of one or more Application Specific Integrated Circuit(s) (ASIC), Field-Programmable Gate Array (FPGA), electronic circuit(s), central processing unit(s), e.g., microprocessor(s) or processors 52, and associated computer readable storage medium, including the memory 54. Instructions embodying a method 100, an example of which is described below with reference to respective FIG. 4, and other methods are executed by the processor 52 from the memory 54, for instance magnetic or optical media, CD-ROM, and/or solid-state/semiconductor memory, e.g., random access memory (RAM) or read-only memory (ROM). Non-transitory components of the memory 54 used herein are capable of storing machine-readable instructions in the form of one or more software or firmware programs or routines, combinational logic circuit(s), input/output circuit(s) and devices, signal conditioning and buffer circuitry and other components that can be accessed by one or more processors to provide a described functionality.

Referring briefly to FIG. 1A, the leak detection system 10 of FIG. 1 may be optionally implemented using stationary components. In other words, none of the components of the leak detection system 10 are configured to move relative to other components. In the illustrated setup for instance, a product 11A in the exemplary form of a welded cold plate having a surface 16A may be positioned relative to the detector 22, with more than one detector 22 being possible in other constructions. As noted above, the emitter 20 may emit one or more beams of the emitted energy 120 toward the surface 16A, with each beam having the same wavelength or wavelength range as the trace gas 18 of FIG. 1. In the non-limiting welded cold plate example of FIG. 1A, the trace gas 18 of FIG. 1 may be inserted into the product 11A as indicated by arrow AA. The trace gas 18 may thereafter flow through an enclosure chamber 140 in the form of a circuitous flow channel formed in the welded cold plate. The trace gas 18 in this construction eventually exits the product 11A as indicated by arrow BB.

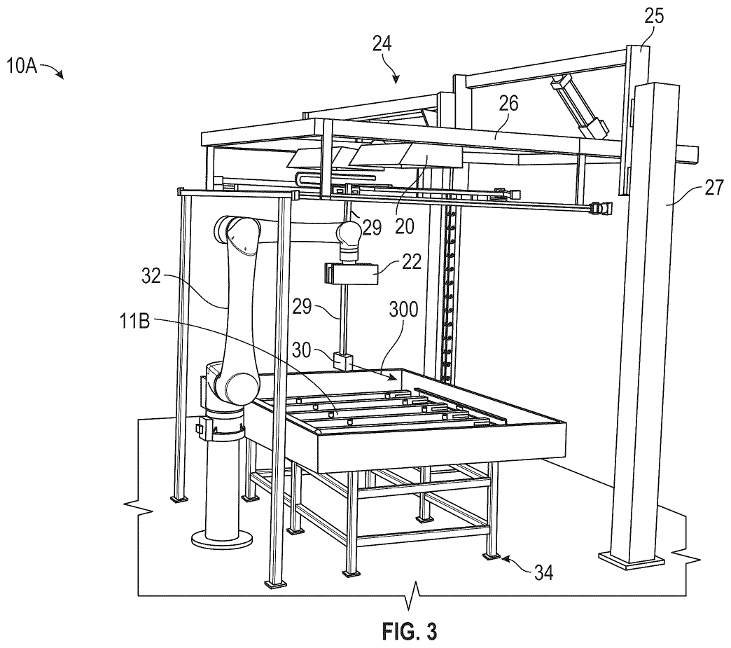

Referring to FIG. 2, the product 11 of FIG. 1 may be alternatively constructed as a battery tray as noted above. Such a product 11B may be positioned on a fixture 34 (see FIG. 3) such as a stationary surface or a moving platform, e.g., a conveyor, in proximity to the above-described emitter 20. In the representative embodiment of FIG. 2, the emitter 20 is a scanning-type emitter, e.g., an IR scanner. Whether constructed as a single device or multiple emitters 20, the emitted energy 120, e.g., a plurality of energy beams as shown, illuminates the product 11B. The reflected energy 120R is thereafter detected by an array of detectors 22A collectively positioned in proximity to the product 11B. Each respective detector 22 of the array of detectors 22A is configured to detect the reflected energy 120R from the product 11B and/or trace gas 18 from a different angle.

FIG. 3 illustrates the leak detection system 10 of FIG. 1 in an alternative leak detection system 10A in which the emitter 20 is configured to move with respect to the product 11, in this case shown as the representative product 11B of FIG. 2. In this construction, the leak detection system 10A may include one or more robots 32, for instance six degree of freedom (6-DOF) industrial robot as shown. The detector 22 in this dynamic implementation is connected to the robot 32, with robot 32 being configured to move the detector 22 as needed with respect to the product 11B, for instance in response to commands from the ECU 50 of FIG. 1. The product 11B may be situated in/on the fixture 34 as shown, with the fixture 34 variously configured as a stationary platform or table, or possibly a manual or automated conveyor in different embodiments.

As part of this approach, or possibly without use of the robot 32, a gantry 24 may be configured to position the emitter 20 relative to the product 11B. The gantry 24 may include various beams 25, horizontal rails 26, and upright support columns 27. While omitted for illustrative simplicity, the gantry 24 would be coupled to a motorized drive unit, a drive belt, or another drive system operable for translating the emitter 20 with respect to the product 11A. The ECU 50 may be tasked with motion control of the gantry 24 and or the robot 32 in different embodiments, or such motion may be controlled by another computer system such as a programmable logic controller, as appreciated in the art.

In a possible construction of the leak detection system 10 of FIG. 1 or 10A of FIG. 3, a three-dimensional (3D) laser scanner 30 may be connected to the gantry 24 via a post 29 as shown, or to the robot 32 or another stationary/static or mobile structure. The 3D laser scanner 30 may be configured to scan the surface 16 (FIG. 1) of the product 11 and thereafter output a 3D scan file 300 to the ECU 50 of FIG. 1. The 3D scan file 300 is indicative of a contour of the surface 16. The ECU 50 may compare the contour of the surface 16 to a calibrated baseline contour, e.g., one recorded in its memory 54 of FIG. 1, to ascertain a surface distortion level of the product 11. The ECU 50 may then generate the electronic output signal 500 of FIG. 1 using the surface distortion level as ascertained using the optional 3D laser scanner 30, for example by accounting for such distortion when locating the leak, or to identify a root cause of the leak.

The present disclosure also lends itself to implementation of an automated reflection-based leak detection method. The ECU 50 of FIG. 1 may be programmed with instructions embodying such a method, with an optional robot-assisted version of the method 100 shown in FIG. 4. FIG. 4 is described below in terms of algorithm code segments or logic blocks for illustrative clarity. Each block is executable from memory 54 by the processor(s) 52 of the ECU 50 unless otherwise noted.

In general, leak detection as set forth herein involves directing the emitted energy 120 from the emitter 20, e.g., of FIG. 1, the emitted energy 120 having a predetermined wavelength range, toward the surface 16 of the product 11. The product 11 defines the enclosure chamber 14, which in turn contains the trace gas 18. Leak detection also includes detecting the reflected energy 120R via the detector(s) 22, with each detector 22 being positioned between the emitter 20 and the product 11 at the offset distance D from the surface 16. The approaches described herein include identifying a detected leak in the product 11 via the ECU 50. This may include comparing the detected spectrum of the reflected energy 120R to a predetermined spectrum of the trace gas 18. The trace gas 18 as described above has a wavelength that falls within the maximum and minimum limits of the predetermined wavelength range of the emitter 20. The ECU 50 then generates the electronic output signal 500 of FIG. 1 in response to the detected leak, the output signal identifying a presence and location of the leak.

Whether assisted by the robot 32 of FIG. 3 or not, the methods contemplated herein may include directing the emitted energy 120 as IR energy having a wavelength in a range of about 750 nm to about 10μ. When the trace gas 18 includes CO₂, for example, this may entail directing the IR energy having a wavelength range of about 2μ to about 10μ, possibly with the offset distance D of about 0.25 m to about 5 m. Directing the emitted energy 120 in the predetermined wavelength range may optionally include directing the emitted energy 120 toward a battery tray, a welded cold plate, or a cover of a vehicle battery in possible usage scenarios as noted above.

FIG. 4 illustrates an embodiment of the method 100 for leak testing the product 11B with the assistance of the robot 32 shown in FIG. 3. After initializing at block B101, e.g., by starting a program on the ECU 50, the method 100 proceeds to block B102.

At block B102, the robot 32 loads the product 11B, i.e., a battery tray in this example, to the fixture 34. Block B102 thus entails placing the product 11B in the leak testing system 10A in preparation for leak testing. The method 100 then proceeds to block B104.

At block B104, the ECU 50 may control a position of the gantry 24 such that the 3D laser scanner 30 is moved to a horizontal overhead position relative to the product 11B. The method 100 then proceeds to block B106.

Block B106 of FIG. 4 entails using the ECU 50 to command the 3D laser scanner 30 to scan the outer perimeter and surface 16 of the product 11B. The 3D laser scanner 30 thereafter outputs the 3D scan file 300 to the ECU 50, with the 3D scan file being indicative of a contour of the surface 16 as noted above. The method 100 then proceeds to block B107.

At block B107, the ECU 50 of FIG. 1 may predict surface distortion of the product 11B based on the contents of the 3D scan file 300. For example, the ECU 50 may compare the contour of the surface 16 to a baseline to ascertain a surface distortion level of the product 11B. Distortion level may be saved and later referenced when determining a location and/or a root cause of detected leaks. The method 100 then proceeds to block B108.

Block B108 of FIG. 4 may include moving the gantry 24 to a vertical position such that motion of the robot 32 is unimpeded. Then, at block B110, the robot 32 may place a lid (not shown) on the fixture 34 for the purposes of the leak test. Once the lid has been placed, the gantry 24 may be commanded to move back to the horizontal position at block B112. The leak test is then ready to commence.

Still referring to FIG. 4, at block B114 the ECU 50 may power on the emitter 20, which in this implementation is positioned directly overhead of the product 11B. Once this occurs, the method 100 proceeds to block B116 where the robot 32 (or a cooperative set of robots or “cobot”) scans the product 11B for leaks using the above-described approach. That is, the detector(s) 22 detect the reflected energy 120R from their position between the emitter 20 and the product 11B. The method 100 then proceeds to block B118.

At block B118, the ECU 50 announces or otherwise identifies the leak location in some manner. Block B118 may include identifying the leak's location on a display screen, for instance, or in a data file, possibly with added audio broadcast. The method 100 thereafter proceeds to block B120.

Block B120 of FIG. 4 includes repair of the identified leak(s). For example, an operator may enter a work cell and repair the identified leak. The method 100 then proceeds to block B121.

Block B121 includes determining whether the identified leaks have been repaired. Options for block B121 include repeating the leak test, for example, or performing another leak test on or offline. The method 100 may repeat block B116 when the leaks have not been repaired, with the method 100 proceeding in the alternative to block B122 when the leaks have been repaired.

At block B122 of the method 100 shown in FIG. 4, the ECU 50 may turn the power off to the emitter 20 before proceeding to block B124. There, the ECU 50 may command the gantry 24 back to the vertical position (analogous to block B108) before proceeding to block B126.

At block B126, the ECU 50 commands the robot 32 to remove the lid that was applied at block B110. The method 100 thereafter proceeds to block B128 where the robot 32 is commanded to remove the product 11B from the fixture 34 of FIG. 3. The method 100 is complete at block B129. Other implementations may be assisted by the robot 32 with or without the assistance of the gantry 24 as will be readily appreciated by those skilled in the art.

The teachings described above with reference to FIGS. 1-4 therefore enable optimal reflection-based imaging for leak detection in a wide variety of applications. In contrast to state of the art leak detection systems, the present leak detection systems 10 and 10A do not require the emitter 20 to be positioned behind the trace gas 18 for radiation absorption before detection by the detector(s) 22. Embodiments such as the FIG. 3 implementation may be robot-assisted, with some components of the leak detection systems 10 and 10A possibly being moveable relative to the product 11. Optional scanning of the product 11 and incorporation of surface contour data indicative of distortion may be used to increase the accuracy of the disclosed leak detection results. These and other potential benefits will be readily appreciated by those skilled in the art in view of the disclosure.

The present disclosure is susceptible of embodiment in many different forms. Representative examples of the disclosure are shown in the drawings and described herein in detail as non-limiting examples of the disclosed principles. To that end, elements and limitations described in the Abstract, Introduction, Summary, and Detailed Description sections, but not explicitly set forth in the claims, should not be incorporated into the claims, singly or collectively, by implication, inference, or otherwise.

For purposes of the present description, unless specifically disclaimed, use of the singular includes the plural and vice versa, the terms “and” and “or” shall be both conjunctive and disjunctive, “any” and “all” shall both mean “any and all”, and the words “including”, “containing”, “comprising”, “having”, and the like shall mean “including without limitation”. Moreover, words of approximation such as “about”, “almost”, “substantially”, “generally”, “approximately”, etc., may be used herein in the sense of “at, near, or nearly at”, or “within 0-5% of”, or “within acceptable manufacturing tolerances”, or logical combinations thereof.

The detailed description and the drawings or figures are supportive and descriptive of the present teachings, but the scope of the present teachings is defined solely by the claims. While some of the best modes and other embodiments for carrying out the present teachings have been described in detail, various alternative designs and embodiments exist for practicing the present teachings defined in the appended claims. Moreover, this disclosure expressly includes combinations and sub-combinations of the elements and features presented above and below.

What is claimed is:

1. A leak detection system comprising:
an emitter configured to direct electromagnetic energy in a predetermined wavelength range toward a surface of a product, the product defining an enclosure chamber that contains a trace gas;
a detector positioned between the emitter and the product at an offset distance from the surface of the product, wherein the detector is configured to detect reflected energy during leak testing of the product; and
an electronic control unit (ECU) in communication with the detector, the ECU being configured to:
receive an electronic input signal from the detector that is indicative of a spectrum of the reflected energy;
identify a detected leak in the product, including comparing the spectrum of the reflected energy to a predetermined spectrum of the trace gas, wherein the trace gas has a wavelength that falls within the predetermined wavelength range of the electromagnetic energy from the emitter; and
generate an output signal in response to the detected leak, the output signal identifying a presence and location of the leak.

2. The leak detection system of claim 1, wherein the emitter includes an infrared (IR) emitter.

3. The leak detection system of claim 2, wherein the predetermined wavelength range of the electromagnetic energy is about 2 microns (μ) to about 10μ.

4. The leak detection system of claim 1, wherein the offset distance from the surface of the product is about 0.25 meters (m) to about 5 m.

5. The leak detection system of claim 1, wherein the detector includes a filter having a bandwidth encompassing the wavelength of the trace gas and the predetermined wavelength range of the electromagnetic energy from the emitter.

6. The leak detection system of claim 1, wherein the ECU is configured to detect the presence and location of the leak by analyzing a difference in contrast between the spectrum of the reflected energy and the predetermined spectrum of the trace gas.

7. The leak detection system of claim 1, wherein the detector includes an array of detectors positioned in proximity to the product, and wherein each respective detector of the array of detectors is configured to detect the reflected energy from a different angle.

8. The leak detection system of claim 1, wherein the emitter is configured to move with respect to the product.

9. The leak detection system of claim 1, further comprising:
a robot, wherein the detector is connected to the robot, and wherein the robot is configured to move the detector with respect to the product.

10. The leak detection system of claim 9, further comprising:
a gantry configured to position the emitter relative to the product.

11. The leak detection system of claim 1, further comprising:
a three-dimensional (3D) laser scanner configured to:
scan the surface of the product;
output a 3D scan file to the ECU that is indicative of a contour of the surface;
compare the contour of the surface to a calibrated baseline contour to ascertain a surface distortion level of the product; and
generate the electronic output signal using the surface distortion level.

12. A leak detection method comprising:
directing electromagnetic energy in a predetermined wavelength range, via an emitter, toward a surface of a product defining an enclosure chamber that contains a trace gas;
detecting reflected energy via a detector that is positioned between the emitter and the product at an offset distance from the surface of the product;
receiving an electronic input signal from the detector via an electronic control unit (ECU), wherein the electronic input signal is indicative of a spectrum of the reflected energy;
identifying a detected leak in the product, via the ECU, including comparing the spectrum of the reflected energy to a predetermined spectrum of the trace gas, the trace gas having a wavelength that falls within the predetermined wavelength range of the electromagnetic energy from the emitter; and
generating an electronic output signal in response to the detected leak, the output signal identifying a presence and location of the leak.

13. The leak detection method of claim 12, wherein:
the emitter includes an infrared (IR) emitter; and
directing the electromagnetic energy in the predetermined wavelength range includes directing IR energy having a wavelength in a range of about 750 nanometers (nm) to about 10 micrometer (μ).

14. The leak detection method of claim 13, wherein:
the trace gas includes carbon dioxide; and
directing the IR energy having the wavelength in the range of about 750 nm to about 10μ includes directing IR energy having a wavelength range of about 2μ to about 10μ.

15. The leak detection method of claim 13, wherein the offset distance from the surface of the product is about 0.25 meters (m) to about 5 m.

16. The leak detection method of claim 12, wherein directing the electromagnetic energy in the predetermined wavelength range includes directing the electromagnetic energy toward a vehicle component.

17. The leak detection method of claim 12, wherein identifying the detected leak includes analyzing a difference in contrast between the spectrum of the reflected electromagnetic energy and the predetermined spectrum of the trace gas.

18. The leak detection method of claim 12, further comprising:
scanning the surface of the product using a three-dimensional (3D) laser scanner;
outputting a 3D scan file to the ECU via the 3D laser scanner, the 3D scan file being indicative of a contour of the surface;
comparing the contour of the surface to a calibrated baseline contour to ascertain a surface distortion level of the product; and
generating the inspection output signal using the surface distortion level.

19. A leak detection system, comprising:
an infrared (IR) emitter array configured to direct IR energy toward a surface of a product in a wavelength range of about 2 microns (μ) to about 10μ, the product defining an enclosure chamber that contains carbon dioxide as a trace gas;
an IR detector array positioned between the IR emitter array and the product at an offset distance of less than about 5 meters (m) from the surface of the product, wherein the IR detector array is configured to detect reflected IR energy during leak testing of the product;
a three-dimensional (3D) laser scanner; and
an electronic control unit in communication with the IR detector array, the ECU being configured to:
receive an electronic input signal from the IR detector array that is indicative of a spectrum of the reflected IR energy;
command the 3D laser scanner to generate a 3D scan file indicative of a contour of the surface;
compare the contour of the surface to a calibrated baseline contour to ascertain a surface distortion level of the product;
identify a detected leak in the product using the surface distortion and by comparing the spectrum of the reflected IR energy to a predetermined spectrum of the trace gas; and
generate an electronic output signal in response to the detected leak, the electronic output signal identifying a presence and location of the leak.

20. The leak detection system of claim 19, further comprising:
a robot configured to move the IR detector array with respect to the product; and
a gantry configured to position the IR emitter array and the 3D laser scanner relative to the product.
